(19) United States
(12) Patent Application Publication
(10) Pub. No.: US 2022/0071183 A1
(43) Pub. Date: Mar. 10, 2022

(54) ANTHELMINTIC LABORATORY ANIMAL MODEL FOR HEARTWORM

(71) Applicant: Zoetis Services LLC, Parsippanty, NJ (US)

(72) Inventors: Brian John Mills, Mattawan, MI (US); Tom L. McTier, Appling, GA (US); Christopher S. Knauer, Kalamazoo, MI (US); Debra Jean Woods, Kalamazoo, MI (US)

(21) Appl. No.: 17/417,829

(22) PCT Filed: Jan. 7, 2020
(86) PCT No.: PCT/US2020/012523

§ 371 Date: Jun. 24, 2021

Related U.S. Application Data
(60) Provisional application No. 62/790,510, filed on Jan. 10, 2019.

(51) Int. Cl. A01K 67/027

(52) U.S. Cl. CPC A01K 67/027; A01K 2227/105; A01K 2267/0337; A01K 2207/20

(57) ABSTRACT

The current invention describes a laboratory animal model for dirofilarial nematodes wherein said laboratory animal is fed a dietary admixture of laboratory feed containing an immunosuppressing agent, for example, a glucocorticoid. The invention also describes the use of the laboratory animal model for screening endoparasiticides for the treatment and/or prevention of filarial nematode infections in animals.

### FIELD OF INVENTION

This invention describes a novel immunosuppressed laboratory animal model for evaluating endoparasiticides for the treatment and/or prevention of dirofilarial nematode infections in animals. The laboratory animal is fed a dietary admixture containing a glucocorticoid or other immunosuppressive agent prior to and after inoculation of dirofilarial third-stage (L3) larvae, particularly Dirofilaria immitis. The immunosuppressed laboratory animal is permissive to the D. immitis infection. As such, the L3 larvae molt into the L4 larvae; the L4 larvae migrate through the subcutaneous tissues and musculature for several months before molting into the immature adult where they enter the blood stream to mature into adult worms that settle in the pulmonary arteries of the heart and lungs. The adult worms become sexually mature and release circulating microfilariae into the blood stream of the host; thereby completing (patency) the infective parasitic cycle.

### BACKGROUND OF INVENTION

Dirofilariasis is a parasitic disease of animals, and occasionally humans, caused by thread-like filarial nematodes, which may result from infection by a species of Dirofilaria such as D. immitis, D. repens, D. tenuis, D. ursi, D. subdermata, D. lutrae, D. striata, and D. spectans.

In dogs, cardiopulmonary dirofilariasis, (i.e., heartworm disease) is caused by Dirofilaria immitis (D. immitis) nematodes. In the case of D. repens, adults are subcutaneous, so while they don't cause heartworm disease per se, they do cause disease/pathology and are prevented with ivermectin doses comparable to those used to prevent heartworm from D. immitis. Dirofilariasis is a severe vector-borne helminthiasis of dogs and cats worldwide. It is transmitted by Culicidae (mosquito) bites and caused by the development of adult D. immitis worms in the heart, pulmonary arteries, and adjacent large blood vessels. Clinically, it leads to irreversible cardiac insufficiency that can result in the death of the animal. Treatment of adult heartworms is clinically challenging; prevention is critical for dirofilarial heartworm control.

The life cycle of D. immitis is well known (McCall et. al., Adv. Parasitol. 66: 193-285, 2008). In brief, a mosquito becomes infected when it draws blood from an infected host (e.g., a dog). The infected blood contains D. immitis microfilariae. In the mosquito, the microfilariae (L1) molt into L2, and then into the infective L3 larval stage, which takes about 10-14 days once the mosquito becomes infected with microfilariae. When the infected mosquito feeds again, it can transmit L3 larvae to a new host (e.g., another dog). In the new host, the L3 larvae develops in the host's subcutaneous tissue for about 3-4 days; after which they then molt into the L4 larvae in the tissues. The parasite remains in the L4 stage for about 2 months after which it molts into the immature adult stage. The immature adults migrate into the host's blood stream from about 70 to 120 days after infection and then move to the right side of the heart, lungs and the pulmonary arteries, where they become mature adults in about 6-7 months. Adult worms produce eggs, which develop in utero into long thin L1 stage larvae (i.e., microfilariae) that are released into the blood stream which can appear about 6-9 months after infection. These microfilaria can be found at concentrations ranging from about several hundred to more than 50,0000 per milliliter of infected blood. The mean length and width of a single D. immitis microfilaria is about 302 μm and 6.3 μm, respectively. A mosquito ingests the circulating microfilariae, when it draws blood from the infected host, and the infective parasitic cycle starts again. Overall, dogs develop chronic microfilaremia starting at 6 to 7 months post-infection, with adult worms surviving up to 7 years. The dog is the definitive host, meaning that the worms mature into adults, mate, and produce offspring while living inside the dog.

D. immitis may be found wherever its vector, the mosquito, and a suitable canine host are found. Mosquitoes of the Culex, Aedes, and Anopheles genera predominate with more than 50 individual species of mosquitoes having been shown to be vectors of D. immitis. Generally, D. immitis may be found on a world-wide basis, but are very common in areas with mild and warm climates.

Macrocyclic lactones (MLs) were introduced more than 30 years ago, yet they remain the only chemical class of drug approved for heartworm disease prevention in the management of D. immitis in veterinary applications. Examples of MLs include, but are not limited to: ivermectin, selamectin, eprinomectin, and includes the milbemycins such as moxidectin, milbemycin, and milbemycin oxime. However, resistance to ML's is common in a variety of parasitic nematodes and appears to be developing in D. immitis.

A number of diagnostic tests have been described for the detection of anthelmintic resistance in nematodes of livestock and horses, including the fecal egg count reduction test, the egg hatch test, microagar larval development test and molecular tests based on benzimidazole resistance (Coles et.al., Vet. Parasitology 136: 167-185, 2006). Prichard et.al., (European patent EP0979278) describes a P-glycoprotein sequence in Haemonchus contortus which may be useful for the diagnosis of ML resistance in gastrointestinal parasitic nematodes. Pritchard, et.al., (U.S. Pat. No. 10,000,811B2) describes markers to predict ML drug resistance in D. immitis. In addition, a number of ML-resistant strains of D. immitis have been identified by phenotypic efficacy testing in dogs (Vet Parasitol.; Bourguinat, C., et.al., 2015, 201(3-4), 167-178; Parasit Vectors, McTier, T. L., et.al., 2017, 10(Suppl 2), 482; and Parasit Vectors, Pulsaki, C. N., et.al., 2014, 7, 494).

Anthelmintic assays have been described for the gastrointestinal nematode, Trichostrongylus colubriformis, in immunosuppressed rats wherein rats were fed a 60 ppm hydrocortisone acetate diet (Veterinary Parasitology, 42, Issues 3-4, 273-279, 1992). ED₉₅ values obtained from the immunosuppressed rat infected with T. colubriformis larvae provided a predictive model for assaying the activity of experimental drugs prior to initiating in vivo studies in ruminants. An immunosuppressed jird (Meriones unguiculatus) model was developed for Haemonchus contortus (J. of Parasitology, 76(2):168-170, 1990). Jirds were fed a 0.02% hydrocortisone diet. Broad spectrum anthelmintics evaluated in the model were uniformly active and comparable to doses required for efficacy in ruminants. Animal studies evaluating vaccine approaches to filariasis have been conducted since the 1940's. These studies have been challenging since different animal models of filariasis, including D. immitis, have distinct life cycles and various degrees of permissiveness (Clin Microbiol Rev. 26(3): 381-421, 2013). Hamsters and jirds are experimental hosts to the related filarid, Acanthocheilonema viteae, from the soft tick Ornithodoros moubata and O. tartakovskyi, and are permissive to infection with transient microfilaremia. Males tend to be more susceptible to infection than females, possibly due to a protective effect imparted by females by 17-β-estradiol and progesterone. Microfilaremia is typically transient, however, some inbred hamster strains develop stable microfilaremia with an assumed normal microfilaria life cycle. Patency commences at 6 to 8 weeks post-infection, peaks at approximately 11-weeks, and declines to undetectable levels by 19-weeks in hamsters; while patency commences at 7 to 10 weeks post-infection in the jird which remains stable for about 2 years. After this time, hamsters are considered “latently” infected, meaning they still harbor adult worms despite being amicrofilaremic. Transfer and immunosuppressive studies suggest that adult worms in latently infected hosts are still capable of producing microfilariae and that latency is most likely due to IgG antibodies that induce antibody-dependent cellular cytotoxicity against microfilariae. As mentioned, the model is permissive and exhibits transient microfilaremia and weak concomitant immunity.

For D. immitis, permissive hosts include the dog and other canids and the ferret. When dogs are experimentally infected, generally more than one-half of infective larvae inoculated will survive to the adult stage. Essentially all dogs experimentally inoculated subcutaneously with L3 larvae will develop a chronic infection and at least 80% of naturally infected dogs develop microfilariae. Sequelae of infection in this model can be both extensive and dire depending on the level of infection. The obstructive presence of adult worms combined with the inflammatory milieu leads to substantial vascular changes, endarteritis, arterial muscular hypertrophy, pulmonary hypertension, pleural effusions, and sometimes death resulting from respiratory distress or cachexia. Other possible complications include eosinophilic pneumonitis, anemia, caval syndrome, and diverse kidney pathology.

Ferrets are also known to be permissive to D. immitis with transient microfilaremia. Both male and female ferrets inoculated with L3 larvae develop chronic infection with adult worms. Although the worms develop to sexual maturity and produce microfilariae by 7 months post-infection, the duration of microfilaremia is short. Cats are considered semi-permissive to D. immitis, as up to about 70% will develop adult infections when given experimental inoculations. However, intensity of infection in cats is usually low with only a few worms, further, microfilariae counts are low and transient in cats. Under prior conditioning, mice and the Lewis rat were shown to be non-permissive hosts to D. immitis.

In many of the models studied, the host develops only transient microfilaremia despite harboring adult worms. This is the nature of latent infections and in most cases is associated with antibodies directed against the sheath of the microfilariae. Therefore, the only robust models for heartworm investigations require the use of ferrets and/or dogs. However, for most investigators, canine studies are simply too long—at least six months—and too expensive to justify exploratory studies outside this proven class of compounds (ML's). Recently, PCT/US2018/017398, described an intermediate model of filarial infection in knock-out mice that would allow researchers to rapidly screen and evaluate novel endoparasiticidal agents without the use of dogs or ferrets. The knock-out mice were described as NOD scid gamma (NOD.Cg-Prkdcscid II2rgtm 1 Wjl/SzJ) immunodeficient mice that can support the infection and development of filarial parasitic worms outside of their definitive hosts. The use of knock-out mice can be an expensive venture and there may or may not be any correlation between infection in the NOD immune-compromised mice and canids. In addition, in these investigations, worms were only allowed to develop to the L4 stage, limiting their use as a complete heartworm model.

Regardless of the use of biological markers for determining drug resistance and transient microfilaremia infection models in hamsters and jirds, and infection models in the dog and ferret; or the use of expensive knock-out immunodeficient mice; there remains a need to develop a laboratory animal model that is robust, inexpensive, and correlates well with known dog and ferret models. The present invention is an immunosuppressed laboratory animal (particularly rodent) model for D. immitis that makes the rodent permissive to the dirofilarial infection; thereby allowing the filarial parasites to circumvent the rodent's defenses and replicate. This immunocompromised rodent model correlates with the dog model. The model provides a patent infection in the rodent wherein the complete parasitic life cycle proceeds from L3 inoculation (infection), then L3 molt to L4 larvae, then L4 molt into immature adult worms, and then maturation to adult breeding worms that release L1 microfilaria back into the hosts blood stream. By using the laboratory immunosuppressed rodent model, fewer dogs are needed to evaluate heartworm preventative compounds, especially for early evaluation of novel heartworm compound libraries. Further, it was shown that the laboratory animal can be used to harvest mature adult worms for further in vitro experimentation and to assess the safety of compounds in animals with existing adult heartworm infections.

### SUMMARY OF THE INVENTION

The immunosuppressed laboratory animal model has proven effective in the investigation of both preventative efficacy and in the assessment of safety in heartworm-infected animals, both in vitro and in vivo, which is of major importance for the development of safe heartworm preventatives for dogs and other animals. This immunosuppressed laboratory animal model is also useful in the investigation of host-parasite interactions, with the potential to identify novel biomarkers for heartworm disease and to contribute to the complex biology of this parasite and its hosts. The use of the immunosuppressed laboratory animal (e.g., rodent) model reduces a) the amount of drug needed for assessment, b) the requirement for the use of regulated species, especially the dog, during early phase discovery research and c) the overall resources required for maintaining larger animal colonies for long periods of time.

In one aspect of the invention, is an immunosuppressed laboratory animal model for dirofilarial nematodes wherein said animal is fed a dietary admixture of an immunosuppressing agent before and after inoculation of dirofilarial L3 larvae. In one aspect, a non-immunosuppressed laboratory animal is fed a dietary admixture of laboratory feed and an immunosuppressing agent to establish and maintain an immunosuppressed laboratory animal. In one aspect, the laboratory animal is a rabbit and the immunosuppressing agent is a glucocorticoid. In another aspect, the laboratory animal is a rodent and the immunosuppressing agent is a glucocorticoid. In another aspect, the laboratory rodent is a rat. In yet another aspect, the laboratory rat is a CD (Sprague Dawley) IGS rat and the immunosuppressing agent is hydrocortisone.

In one aspect of the invention, the glucocorticoid is selected from the group consisting of hydrocortisone, a hydrocortisone salt, methylprednisolone, prednisolone, prednisone, and triamcinolone. The preferred glucocorticoid is hydrocortisone or a hydrocortisone salt thereof. The hydrocortisone salts include, but are not limited to: acetate, butyrate, hemi-succinate, sodium phosphate, sodium succinate, and valerate. The preferred hydrocortisone salt is acetate.

In another aspect of the invention, the dirofilarial nematode is Dirofilaria immitis (D. immitis) or Dirofilaria repens (D. repens). The preferred dirofilarial nematode is D. immitis.

In one aspect of the invention, is an immunosuppressed laboratory animal model for dirofilarial nematodes wherein said animal is fed a dietary admixture of a laboratory animal feed containing an immunosuppressing agent before and after inoculation of dirofilarial L3 larvae; wherein said animal is a rat and the immunosuppressing agent is hydrocortisone acetate and the dirofilarial L3 larvae is D. immitis.

In one aspect of the invention, the dietary admixture of laboratory animal feed contains about 20 ppm to 250 ppm of a glucocorticoid. In another aspect, the dietary admixture contains about 40 to 200 ppm of a glucocorticoid. In another aspect, the dietary admixture contains about 50, 75, 100, 125, 150, 175, or 200 ppm of a glucocorticoid. In another aspect, the dietary admixture can contain intermittent amounts of a glucocorticoid, for example, 60 ppm, 90 ppm, 115 ppm, 130 ppm, 140 ppm, 165 ppm, 180 ppm, 190 ppm, and the like. In one aspect, the preferred glucocorticoid is hydrocortisone.

In another aspect of the invention, the dietary admixture of laboratory animal feed contains about 20 ppm to 250 ppm hydrocortisone. In another aspect, the dietary admixture contains about 40 to 200 ppm of hydrocortisone. In another aspect, the dietary admixture contains about 50, 75, 100, 125, 150, 175, or 200 ppm of hydrocortisone. In another aspect, the dietary admixture can contain intermittent amounts of hydrocortisone, for example, 60 ppm, 90 ppm, 115 ppm, 130 ppm, 140 ppm, 165 ppm, 180 ppm, 190 ppm, and the like. In yet another aspect, the dietary admixture contains about 50 ppm hydrocortisone. In another aspect, the dietary admixture contains about 75 ppm hydrocortisone. In another aspect, the dietary admixture contains about 100 ppm hydrocortisone. In another aspect, the dietary admixture contains about 150 ppm hydrocortisone. In another aspect, the dietary admixture contains about 200 ppm hydrocortisone. Preferably, hydrocortisone acetate is admixed with the laboratory animal feed to provide the calculated immunosuppressing amount of hydrocortisone. The dietary admixture is fed to the animal ad libitum.

In one aspect of the invention, the dietary admixture of laboratory animal feed contains about 200 ppm hydrocortisone that is administered to the rodent for at least 3 days prior to inoculation with D. immitis L3 larvae. In another aspect, the dietary admixture containing about 200 ppm hydrocortisone is administered to the rodent for about 5 to 10 days prior to inoculation with D. immitis L3 larvae. In another aspect, the dietary admixture containing about 200 ppm hydrocortisone is administered to the rodent for about 7 to 9 days prior to inoculation with D. immitis L3 larvae. In another aspect, the dietary admixture containing about 200 ppm hydrocortisone is administered to the rodent for about 8 days prior to inoculation with D. immitis L3 larvae. On or about the 9^{th} day, the rodent is fed the 200 ppm hydrocortisone diet and is inoculated with D. immitis L3 larvae. Following L3 inoculation, the rodent is fed the 200 ppm hydrocortisone diet for a period of time and then the amount of hydrocortisone can be reduced to about 50 ppm through necropsy. Necropsy can be performed after the immature adult worms have developed and migrated to the heart after about 70 to about 120 days after L3 inoculation, and preferably at about 90 to about 110 days after L3 inoculation. These immature adult worms can be harvested from the rodent and used for in vitro and in vivo heartworm studies. In addition, necropsy can be performed after the immature adult worms have matured and are producing and releasing circulating microfilariae into the blood stream at about 180 days to about 260 days after L3 inoculation. The immunosuppression diet can continue further beyond the 260 days, particularly if adult worms are to be maintained in the animal to produce circulating microfilaria for harvesting or for further study.

In another aspect, the rodent is administered the dietary admixture of laboratory animal feed containing about 200 ppm hydrocortisone for at least 3 days after L3 inoculation. In another aspect, the rodent is administered the dietary admixture containing about 200 ppm hydrocortisone for at least 5 to about 20 days after L3 inoculation. In another aspect, the rodent is administered the dietary admixture containing about 200 ppm hydrocortisone for at least 8 to about 14 days after L3 inoculation. In another aspect, the rodent is administered the dietary admixture containing about 200 ppm hydrocortisone for about 12 days after L3 inoculation.

In another aspect, the rodent is administered the dietary admixture containing about 200 ppm hydrocortisone for about 21 days.

Following this pre- and post-inoculation period wherein the rodent was administered about 200 ppm hydrocortisone in a dietary admixture; the amount of hydrocortisone on a ppm basis per day was reduced for subsequent feeding periods. The reduced daily amount of hydrocortisone in the dietary admixture can range from about 25 ppm to about 100 ppm. The preferred reduced amount of hydrocortisone is about 35 ppm to about 75 ppm, and more preferably, about 50 ppm. This reduced amount of hydrocortisone is administered daily in the dietary feed admixture for at least 60 days through necropsy. The preferred reduced amount of hydrocortisone is administered daily in the dietary feed admixture for at least 70 days through necropsy.

In another aspect, the rodent is administered the dietary admixture containing about 200 ppm hydrocortisone for at least 3 days to about 8 days prior to inoculation with D. immitis L3 larvae; administered the same 200 ppm hydrocortisone diet on the day of L3 inoculation (i.e., Day 9); and then administered the same 200 ppm hydrocortisone diet for at least another 5 days to 20 days, and preferably for another 8 days to about 14 days, and more preferably, for about 12 days. On or about the 22^{nd} day of immunosuppression dosing, the rodent is then administered a dietary admixture containing about 50 ppm hydrocortisone. The 50 ppm hydrocortisone dose is administered for at least 60 days through necropsy. Preferably, the 50 ppm hydrocortisone dose is administered for at least 70 days through necropsy. More preferably, the 50 ppm hydrocortisone dose is administered for about 94 days prior to necropsy. After about 70 days to about 120 days post-inoculation, the rodent can be necropsied and immature adult worms harvested for use in further in vitro and in vivo studies. Preferably, the immature adult worms are harvested at about 90 to about 110 days post-inoculation.

In another aspect, the rodent is administered the dietary admixture containing about 200 ppm hydrocortisone for about 8 days prior to inoculation with D. immitis L3 larvae; administered the same 200 ppm hydrocortisone diet on the day (i.e., Day 9) of L3 inoculation; and then administered the same 200 ppm hydrocortisone diet for about more 12 days. On or about the 22nd day of immunosuppression dosing, the rodent is then administered a dietary admixture containing about 50 ppm hydrocortisone. The 50 ppm hydrocortisone dose is administered for about 94 days prior to necropsy to harvest the immature adult worms.

In another aspect, the rodent is administered the dietary admixture containing about 200 ppm hydrocortisone for at least 3 to about 8 days prior to inoculation with D. immitis L3 larvae; administered the same 200 ppm hydrocortisone diet on the day of L3 inoculation; and then administered the same 200 ppm hydrocortisone diet for at least another 8 days to about 14 days, and preferably, for about 12 days. On or about the 22nd day of immunosuppression dosing, the rodent is then administered a dietary admixture containing about 50 ppm hydrocortisone. The 50 ppm hydrocortisone dose is administered for at least 70 days through necropsy. After about 180 days to about 260 days of 50 ppm hydrocortisone dosing, the rodent can be necropsied and the mature adult worms harvested for use in further in vitro and in vivo studies; and the circulating microfilariae can also be used. In another aspect, the 50 ppm hydrocortisone dose is administered for more than 260 days to acquire the mature worms as described above. These later stage worms can be used for other in vitro and in vivo studies.

In another aspect of the invention, is an immunosuppressed laboratory animal model for dirofilarial heartworm nematodes wherein said animal is fed a dietary admixture of an immunosuppressing agent before and after inoculation of dirofilarial L3 larvae. In another aspect, is an immunosuppressed laboratory animal model for dirofilarial heartworm nematodes wherein said animal is fed a dietary admixture of an immunosuppressing agent before and after inoculation of dirofilarial L3 larvae wherein the immunosuppressing agent is a glucocorticoid and the laboratory animal is a rodent. In another aspect, is an immunosuppressed rodent model for dirofilarial heartworm nematodes wherein said rodent is fed a dietary admixture comprising the immunosuppressing agent hydrocortisone before and after inoculation of dirofilarial L3 larvae wherein dirofilarial heartworm nematode is D. immitis or D. repens. In another aspect, is an immunosuppressed rodent model for dirofilarial heartworm nematodes wherein said rodent is a rat and is fed a dietary admixture comprising about 20 ppm to 250 ppm of the immunosuppressing agent hydrocortisone before and after inoculation of dirofilarial L3 larvae wherein dirofilarial heartworm nematode is D. immitis. In another aspect, is an immunosuppressed rat model for dirofilarial heartworm nematodes wherein said rat is fed a dietary admixture comprising about 50 ppm to 200 ppm of the immunosuppressing agent hydrocortisone before and after inoculation of D. immitis dirofilarial L3 larvae. In another aspect, is an immunosuppressed rat model for dirofilarial heartworm nematodes wherein said rat is fed a dietary admixture comprising about 200 ppm hydrocortisone for at least three days before inoculation with D. immitis dirofilarial L3 larvae and then fed the 200 ppm hydrocortisone dietary admixture for about 8 to 14 days after inoculation. In another aspect, is an immunosuppressed rat model for dirofilarial heartworm nematodes wherein said rat is fed a dietary admixture comprising about 200 ppm hydrocortisone for at least three days before inoculation with D. immitis dirofilarial L3 larvae and then fed the 200 ppm hydrocortisone dietary admixture for about 8 to 14 days after inoculation and subsequently fed a 50 ppm hydrocortisone dietary admixture for at least 70 days through necropsy.

In one aspect of the invention, is a method of screening endoparasitic agents for the prevention and/or treatment of dirofilarial nematodes, and preferably D. immitis, in animals using the immunosuppressed laboratory animal (e.g., rodents) model. In one aspect of the invention, the endoparasitic agent includes, but is not limited to: macrocyclic lactones (for example, selamectin, ivermectin, eprinomectin, and the like; the milbemycins (e.g., moxidectin, milbemycin, milbemycin oxime, and the like); cyclooctadepsipeptides (e.g., emodepside, compounds disclosed in U.S. Pat. Nos. 5,514,773; 5,747,448; 5,646,244; and 5,874,530; PCT publications WO2016/187534 and WO2017/116702), and PCT applications (PCT/US2018/62749) and PCT/US2019/031158). In yet another aspect, the immunosuppressed laboratory animal (e.g., rodents) model can be used to screen anti-filarial endoparasitic agents for potential D. immitis heartworm prevention and/or treatment in canines. In another aspect, is a method of continuing the patency of D. immitis nematodes in the immunocompromised rodent model for further adult worm harvesting for further endoparasiticidal screening studies and for transplantation into other animals for further studies.

### Definitions

For purposes of the present invention, as described and claimed herein, the following terms and phrases are defined as follows:

“About”, as used herein, refers to the indicated value of the variable and to all values of the variable that are within the experimental error of the indicated value (e.g., within the 95% confidence interval for the mean) or within 10 percent of the indicated value, whichever is greater. In each instance, the term “about” can be considered as an optional term that can be interpreted as being absent from the sentence so that the day ranges are as defined.

“Dietary admixture”, as used herein, unless otherwise indicated, refers to a laboratory animal feed (e.g., Purina Rodent Laboratory Chow, LabDiet Rodent 5001 and 5002; and Animal Specialties & Provisions Rodent LabDiet) admixed with an immunosuppressing agent.

“Endoparasitic agent”, as used herein, unless otherwise indicated, refers to veterinary and pharmaceutical compounds (drugs) that when administered to an animal prevents and/or treats said animal infected with a filarial nematode; and in particular, canids.

“Immunosuppressing agent(s)”, as used herein, unless otherwise indicated, refers to glucocorticoids (corticosteroids), e.g., hydrocortisone, cortisol, prednisolone, betamethasone, and the like; immunomodulating hormones, e.g., estrogen, progesterone, epinephrine, and the like; alkylating agents (e.g., cyclophosphamide, nitrosoureas, platinum compounds); antimetabolites (e.g., folic acid analogues (e.g., methotrexate), purine analogues (e.g., azathioprine and mercaptopurine), pyrimidine analogues (e.g., fluorouracil), and protein synthesis inhibitors; cytotoxic antibiotics (e.g., dactinomycin, anthracyclines, mitomycin C, bleomycin, mithramycin, and the like); and immunomodulating drugs like ciclosporin, tacrolimus, sirolimus, and everolimus.

“Inoculation” as used herein, unless otherwise indicated, refers to the host (e.g., rat) being infected with dirofilarial nematode L3 or L4 larvae, or adult (immature or mature) worms.

“Laboratory animal(s)”, as used herein, unless otherwise indicated, refers to rabbits and rodents. The preferred laboratory animal is a rodent. The preferred rodent is a rat.

“Parasite(s)”, as used herein, unless otherwise indicated, refers to endoparasites. Endoparasites are parasites that live within the body of its host and include helminths (e.g., trematodes, cestodes, and nematodes) and protozoa. Preferred endoparasites are dirofilarial nematodes. A more preferred dirofilarial nematode is Dirofilaria immitis.

“Rodent(s)”, as used herein, unless otherwise indicated, refers to laboratory animals and includes mice, rats, jirds, hamsters, and guinea pigs. Preferred rodents are mice, rats, and jirds. More preferred rodents are rats, and in particular, the CD (Sprague Dawley) IGS rat. The CD (Sprague Dawley) IGS rat is a registered trademarked (CD®) rat from Charles River Laboratories (Wilmington, Mass., USA; nomenclature: Crl:CD(SD)). CD refers to caesarean-derived and IGS refers to International Genetic Standardization which is a management program used to minimize inbreeding and control random genetic drift that would otherwise lead to colony divergence among colonies bred in different locations worldwide.

“Treatment”, “treating”, and/or to “treat” all refer to reversing, alleviating, or inhibiting the endoparasitic infection or condition. As used herein, these terms also encompass, depending on the condition of the animal, preventing the onset of a disorder or condition, or of symptoms associated with a disorder or condition, including reducing the severity of a disorder or condition or symptoms associated therewith prior to affliction with said infection or infestation or after said infection or infestation. In the case of D. immitis, to treat refers to the prevention of the L3 larvae to molt into the L4 stage and/or the L4 larvae from molting into the immature adult worms, and subsequently, mature adult worms.

As used herein, percent of components of the composition refers to percentages of the total weight of the dietary admixture and is referred to as “% w/w” or “w/w %” which defines the mass fraction of the compositional component expressed as a percentage, determined according to the formula mi/mtot×100, wherein mi is the mass of the substance of interest present in the composition, and mtot is the total mass of the composition.

As used herein, “at least” refers to one or more days, weeks, or months. For Example, at least 3 days prior to inoculation refers to 3 days, 4, days, 7 days, and more, prior to inoculation. Use of the term “at least” also refers to the number of days after inoculation.

As used herein, “ppm” refers to parts per million.

As used herein, “L3” refers to the larval stage of a dirofilarial nematode, for example, Dirofilaria immitis, D. repens, D. tenuis, D. ursi, D. subdermata, D. lutrae, D. striata, and D. spectans. The preferred L3 larvae is D. immitis.

### DETAILED DESCRIPTION

The current immunosuppressed laboratory animal model, particularly rodent model, was developed by evaluating different doses of hydrocortisone using a dietary admixture of animal feed and hydrocortisone acetate fed to rodents ad libitum prior to and after inoculation with D. immitis L3 larvae. Additional in vivo studies have investigated other parameters, such as non-immunosuppression, rat age and gender, single and multiple heartworm inoculation levels and post-inoculation durations. Infected rats were maintained for nearly 7 months when up to 8 adult worms 6″ inches in length were recovered and microfilariae observed, confirming successful life cycle completion to fecundity. For D. immitis rat model efficacy validation, oral efficacy of compounds representing multiple antiparasitic classes, including ivermectin, moxidectin, emodepside, novel cyclooctadepsipeptides, an isoxazoline, and a bisimide were evaluated.

Although the heartworm life cycle is similar in the rat, ferret and dog, we have determined that preventative efficacy can be assessed 1 month earlier in the rat (˜106 days) than in the ferret and dog (˜140 days). The macrocyclic lactones, ivermectin and moxidectin, which are currently used in heartworm preventive therapies, achieved 96.5% and 100% efficacy at their use-dose. Emodepside and some cycloocta-depsipeptide analogs also demonstrated 100% prevention of heartworm.

The model has also been adapted to support evaluation of safety in adult heartworm infected dogs, both with an in vivo model and by providing adult heartworms for in vitro testing. Approximately 4000 adult heartworms have been generated for in vitro testing of over 200 compounds. This has substantially reduced the number of heavily infected dogs required to obtain a similar number of worms. Ongoing model optimization has resulted in further reduction of the immunosuppression regimen to the minimum level needed to ensure optimal infection and overall rodent health. In addition, heartworms from rat can be excised and transferred (transplanted) into a dog for in vivo safety assessments with heartworm endoparasiticides.

Laboratory animal models are an essential component in the discovery of new drugs reducing the need for testing in higher order target species, which often cannot sustain the number of compounds and evaluations necessary for early triage in lead seeking and lead optimization. The novel rodent heartworm model has demonstrated good correlation with the dog prevention efficacy model and the capability to reduce the number of dogs required to evaluate compounds both in vitro and in vivo, while shortening timelines for key decisions. There are different laboratory strains of rat and include the non-limiting examples: Wistar rat, Sprague-Dawley rat, including the CD (Sprague Dawley) IGS rat, Hairless rat, Long-Evans rat, Wistar Han IGS rat, Brown Norway rat, Copenhagen rat, Fischer rat, F344 rat, and the Lewis rat. The CD (Sprague Dawley) IGS rat is a preferred rat due to its weight and size characteristics.

Immunomodulating agents include corticosteroids. Corticosteroids are involved in a wide range of physiological processes, including stress response, immune response, regulation of inflammation, carbohydrate metabolism, protein catabolism, blood electrolyte levels, and behavior. Corticosteroids down regulate the immune system and affect white blood cell functionality. Corticosteroids are synthetic drugs that closely resemble cortisol, a naturally occurring hormone produced by mammalian adrenal glands. By chemical structure, the corticosteroids are classed into different groups. Group A is defined as hydrocortisone type corticosteroids that include hydrocortisone and its respective salts (e.g., acetate, butyrate, hem i-succinate, sodium phosphate, sodium succinate, and valerate), loteprednol etabonate, betamethasone, dexamethasone, fluorometholone, methylprednisolone, prednisolone, prednisone, rimexolone, cortisol, and triamcinolone. Group B are defined as acetonides, and include, for example, amcinonide, budesonide, desonide, fluocinolone, fluocinonide, halcinonide, and triamcinolone acetonide. Group C are defined as betamethasone types, and include, for example, beclomethasone, betamethasone, dexamethasone, fluocortolone, halometasone, and mometasone. Corticosteroid refers to both the glucocorticoids and mineralocorticoids. The preferred corticosteroids are glucocorticoids that modulate inflammation and the immune system. The term glucocorticoid is a portmanteau (glucose+cortex (adrenal)+steroid) composed from its role in the regulation of glucose metabolism, synthesis in the adrenal cortex (cortisol) and its steroid structure. The preferred glucocorticoid is hydrocortisone and the hydrocortisone salts thereof, prednisolone, and dexamethasone. The more preferred glucocorticoid is hydrocortisone and hydrocortisone salts thereof. The even more preferred glucocorticoid is hydrocortisone acetate. In particular, the more preferred glucocorticoid is hydrocortisone-21-acetate. The manipulation of the immune system in the rodent in this way does not appear to have affected the function of known heartworm preventives in delivering similar efficacies as is found in similar studies using the dog model, offering confidence that this rat model will be predictive of novel compounds that will provide similar preventive heartworm efficacy in the dog.

In addition to glucocorticoids, other immunomodulating agents can be used in the laboratory animal model. These additional immunomodulating agents include hormones that include, but are not limited to estrogen, progesterone, androgen, progestin, testosterone, epinephrine, and dehydroepiandrosterone (DHEA). In addition, immunosuppressive agents, including, but not limited to alkylating agents (e.g., cyclophosphamide, nitrosoureas, platinum compounds); antimetabolites (e.g., folic acid analogues (e.g., methotrexate), purine analogues (e.g., azathioprine and mercaptopurine), pyrimidine analogues (e.g., fluorouracil), and protein synthesis inhibitors; cytotoxic antibiotics (e.g., dactinomycin, anthracyclines, mitomycin C, bleomycin, mithramycin, and the like); and immunomodulating drugs like ciclosporin, tacrolimus, sirolimus, and everolimus.

The immunosuppressing agent can be administered to the laboratory animal, particularly rodents, orally, topically, and by injection (intramuscular, subcutaneously, and intravenously). Oral routes of administration are preferred routes and include gavage and dietary admixture. The preferred route is by dietary admixture.

The dietary feed admixture was prepared in a two-step process. First, a Type A concentrated mixture (20,000 ppm; 2% w/w) of hydrocortisone was prepared. To about 977.22 g (˜98% w/w) LabDiet 5002 rodent (meal), 22.78 grams of hydrocortisone acetate was added (˜20 g hydrocortisone). The rodent meal and hydrocortisone acetate were mixed uniformly. Second, a portion (or all) of the concentrated hydrocortisone mixture was mixed with more LabDiet 5002 rodent feed in a mixer for about 20 minutes. Once uniform, water (10% w/w) was added to the mixture and mixed for an additional 10 minutes. The wet mixture was pelletized and dried. The pelleted dietary admixture contains about 200 ppm hydrocortisone. Lesser (e.g., 50 ppm) and greater (e.g. 250 ppm) amounts of ready to feed dietary admixture pellets were made by adjusting the amount of the concentrated hydrocortisone mixture that was added to the feed. Immunosuppression dosing is based on the amount of hydrocortisone in the feed admixture. The molecular weight of hydrocortisone is 362.46 g/mol and the molecular weight of hydrocortisone acetate is 404.5 g/mol. Therefore, about 1.116 mg of hydrocortisone acetate is added to about 998.884 g of feed to prepare a 1 mg/kg (˜1 ppm) dietary admixture of hydrocortisone.

Upon receipt, newly acquired rats are fed a normal laboratory rat chow diet for a few days for acclimation. Once the immunosuppression diet begins, rats can be fed a glucocorticoid (e.g., hydrocortisone) dietary admixture for about 130 days using at least two dosing concentrations at about 50 ppm, 75 ppm, 100 ppm, 125 ppm, 150 ppm, 175 ppm, 200 ppm, 225 ppm, and 250 ppm. Intermittent dose amounts of a glucocorticoid (e.g., hydrocortisone) also include, for example, 65 ppm, 80 ppm, 115 ppm, 135 ppm, 160 ppm, 180 ppm, 190 ppm, 210 ppm, 230 ppm, and 245 ppm, and the like.

Earlier studies showed that rats fed a 200 ppm hydrocortisone diet for about 120 days had an increased incidence of mortality. Therefore, to maintain normal rat growth, health, and behavior, the 200 ppm hydrocortisone diet should be fed for a duration of about 85 days; preferably about 60 days, and more preferably about 30 days, and even more preferably about 21 days. This 200 ppm dietary duration includes the pre-L3 inoculation period, L3 inoculation day, and the post-L3 inoculation period. Thereafter, rats can be fed the immunosuppressant diet with a reduced amount of hydrocortisone at about 20 ppm, 30 ppm, 40 ppm, 50 ppm, or 60 ppm through necropsy. The preferred reduced amount of hydrocortisone is about 50 ppm. Rats should be maintained on the immunosuppressant diet containing about 50 ppm hydrocortisone for at least 60 days through necropsy, and preferably for at least 70 days through necropsy. A preferred immunosuppression diet plan includes feeding the rodent a 200 ppm hydrocortisone dietary admixture for about 8 days prior to D. immitis L3 inoculation; then continue feeding the rat the same 200 ppm hydrocortisone dietary admixture for about 13 days post-inoculation (including inoculation day); then continue feeding the rat a 50 ppm hydrocortisone dietary admixture for at least 60 days through necropsy, and preferably for at least 70 days through necropsy. Preferably, the rodent is fed the 50 ppm hydrocortisone dietary admixture for about 90 days to about 100 days, and preferably about 94 days, before necropsy, which allows the immature adult worms to migrate to the heart and lungs, where they can be more easily harvested. A longer duration (about 180 to about 260 days post L3 inoculation) of the 50 ppm hydrocortisone diet can be fed to the rat to allow the immature adult worms to mature and begin producing and releasing microfilariae (L1) into the blood stream. In addition, the reduced amount of hydrocortisone can be administered within the dietary admixture for greater than 260 days post-inoculation to maintain the mature adult worms in vivo for future studies.

Heparinized blood from infected dogs with patent adult D. immitis worms and circulating D. immitis microfilaria were obtained and membrane fed to Aedes aegypti mosquitoes. The infected blood contained about 50-100 microfilariae/20 μL. After about 15-17 days following feeding, mosquitoes were collected and crushed to obtain the infective L3 larvae. Larvae were stored in a Roswell Park Memorial Institute (RPMI) medium which is a cell and tissue culture medium used for growing mammalian cell lines. The RPMI is a nutrient blend of amino acids, vitamins, organic and inorganic supplements, and salts. The L3 larvae were injected into the rat in about a 0.2 mL volume of the RPMI culture media.

D. immitis antigen can be monitored using an enzyme-linked immunosorbent assay (ELISA) for the detection of antigen to adult D. immitis antigen in canine and feline plasma or serum. For example, the DiroCHECK™ test kit can be used to detect the D. immitis antigen, particularly from adult female worms. Other IgG antibody and serological tests can be used to monitor for the antigen(s). In addition, polymerase chain reaction (PCR) tests can be performed to further substantiate Dirofilaria spp.

Dirofilaria is a genus of nematodes, or roundworms, in the family Onchocercidae. Some species cause dirofilariasis, a state of parasitic infection, in humans and animals. There are about 27 species in the genus. These are generally divided into two subgenera, Dirofilaria and Nochtiella. Some species are well-known parasites, including D. immitis, the dog heartworm, Dirofilaria repens, which affects many types of nonhuman mammals, and Dirofilaria tenuis, which usually parasitizes raccoons, but can infect humans, as well. Human dirofilariasis is generally caused by D. immitis and D. repens. The former can cause pulmonary dirofilariasis, which may have no symptoms. Another form of the infection can be characterized by a painful lump under the skin or infection of the eye. The nematode infection is spread by mosquitoes. Species in the genus include: D. acutiuscula, D. aethiops, D. ailure, D. asymmetrica, D. cancrivori, D. conjunctivae, D. corynodes, D. desportesi, D. fausti, D. freitasi, D. genettae, D. hystrix, D. immitis (dog heartworm), D. indica, D. louisianensis, D. macacae, D. macrodemos, D. magalhaesi, D. magnilarvata, D. pongoi, D. reconditum, D. repens, D. timidi, D. uniformis, D. ursi, D. tawila, and D. tenuis. The preferred dirofilarial nematodea are D. immitis and D. repens. The more preferred dirofilarial nematode is D. immitis.

### Examples

In an early study, thirty-two male CD (Sprague Dawley) IGS rats weighing 175-250 grams were randomized into 4 treatment groups (n=8/group). Each group received a medicated diet containing hydrocortisone acetate. T01, T02, and T03 received a 200 ppm, 100 ppm, and 50 ppm hydrocortisone dose, respectively, for 120 days. The fourth group, T04, received a 200 ppm dose through Day 17; subsequent dosing through necropsy was with 50 ppm hydrocortisone. Feed was administered ad-libitum. On Day 6, animals were inoculated with approximately 15 L3 D. immitis larvae (Michigan strain) by subcutaneous injection into the inguinal area. On Day 114 post-inoculation, rats were necropsied and adult worm counts assessed. In total, about 3, 11, 5, and 19 adult worms were collected from the T01, T02, T03, and T04 groups, respectively.

In a second study, duration of immunosuppression and timing of inoculation was assessed to establish criteria for consistent adult dog heartworm infections in the rat. Male and female CD (Sprague Dawley) IGS rats weighing 40-50 grams (Group A) and 151-175 grams (Group B) were used in the study. The study design is described below in Table 1.

TABLE 1
Study Design for the duration of immunosuppression
and timing of D. immitis inoculation in rats

| | | | | | | Day |
|---|---|---|---|---|---|---|
| | | L3 | Days of | Days of | | post- |
| | # of | inocu- | immuno- | immuno- | Nec- | inocu- |
| | animals | lation | suppression | suppression | ropsy | lation |
| Group | (gender) | day | 200 ppm | 50 ppm | day | (±1) |
| T01 (A) | 6M | 0 | None | None | 98 | 98 |
| T02 (A) | 6M | 0 | None | None | 112 | 112 |
| T03 (A) | 6M | 0 | None | None | 126 | 126 |
| T04 (A) | 6M | 0 | None | None | 140 | 140 |
| T05 (A) | 6M | 0 | −8 to 7 | 7-98 | 98 | 98 |
| T06 (A) | 6M | 0 | −8 to 7 | 7-112 | 112 | 112 |
| T07 (A) | 6F | 0 | −8 to 7 | 7-112 | 112 | 112 |
| T08 (A) | 6M | 0 | −8 to 7 | 7-126 | 126 | 126 |
| T09 (A) | 6M | 0 | −8 to 7 | 7-140 | 140 | 140 |
| T10 (A) | 6M | 7 | −8 to 21 | 21-98 | 105 | 98 |
| T11 (A) | 6M | 7 | −8 to 21 | 21-112 | 119 | 112 |
| T12 (A) | 6M | 7 | −8 to 21 | 21-126 | 133 | 126 |
| T13 (A) | 6M | 7 | −8 to 21 | 21-140 | 147 | 140 |
| T14 (B) | 6M | 7 | −8 to 21 | 21-98 | 105 | 98 |
| T15 (B) | 6M | 7 | −8 to 21 | 21-112 | 119 | 112 |
| T16 (B) | 6F | 7 | −8 to 21 | 21-112 | 119 | 112 |
| T17 (B) | 6M | 7 | −8 to 21 | 21-126 | 133 | 126 |
| T18 (B) | 6M | 7 | −8 to 21 | 21-140 | 147 | 140 |

On Day −8, medicated hydrocortisone diet at 200 ppm was provided to treatment groups T05-T18. On Day 0, treatment groups T01-T09 received an inoculation of approximately 50 L3 D. immitis larvae (Michigan strain) in 0.2 mL RPMI, subcutaneously injected into the inguinal area. On Day 7, T10-T18 received an inoculation of approximately 50 (2×25) L3 D. immitis by subcutaneous injection into the inguinal area. On Day 7, Groups T05-T09 were converted from the 200 ppm hydrocortisone diet to the 50 ppm hydrocortisone diet. On Day 21, groups T10-T18 were converted from the 200 ppm hydrocortisone diet to the 50 ppm hydrocortisone diet. Diet for all groups was administered ad-libitum. Necropsy occurred on Days 98, 112, 126 and 140 (±1) post-inoculation. Blood was obtained at necropsy to test for D. immitis antigen using the DiroCHECK ELISA based serological test. Data from this study is presented below in Tables 2 and 3.

TABLE 2
Study results for the duration of immunosuppression
and timing of D. immitis inoculation in rats.

| | Mean worm | Percent of | Worm counts |
|---|---|---|---|
| | count | rats with | from body |
| Group | (range) | infection (n) | cavity digestion |
| T01 | 0 (0) | 0 (6) | 0 |
| T02 | 0 (0) | 0 (6) | 0 |
| T03 | 0.3 (0-2) | 17 (6) | 0 |
| T04 | 0 (0) | 0 (6) | 0 |
| T05 | 4.2 (2-8) | 100 (5) | 0 |
| T06 | 6.0 (5-7) | 100 (5) | 2 |
| T07 | 0 (0) | 0 (6) | 0 |
| T08 | 5.3 (4-11) | 100 (4) | 7 |
| T09 | 2.4 (0-6) | 60 (5) | 5 |
| T10 | 2.2 (2-7) | 60 (5) | 0 |
| T11 | 5.5 (2-11) | 100 (4) | 2 |
| T12 | 5.3 (0-9) | 83 (6) | 4 |
| T13 | 4.0 (3-6) | 100 (4) | 4 |
| T14 | 3.6 (0-6) | 83 (6) | 0 |
| T15 | 5.7 (2-8) | 100 (6) | 5 |
| T16 | 0.2 (0-1) | 17 (6) | 0 |
| T17 | 7.3 (2-16) | 100 (6) | 8 |
| T18 | 7.7 (4-18) | 100 (6) | 14 |

As can be seen in Table 2, only 1 non-immunosuppressed rat had worms (T06) showing immunosuppression is necessary to establish meaningful worm burdens for studies. Female rats were almost wormless clearly demonstrating a gender component in rats causing a non-permissive state, or perhaps, a semi-permissive state. For the male immunosuppressed groups, 96% of mature rats with an average of 5.9 worms and 87% of newly weaned rats with an average of 4.3 worms was achieved. Extending the 200 ppm diet duration to or after inoculation did not have a significant positive or negative effect. An increased dual inoculation challenge produced an increase in worm burden and a greater percentage of infected rats. Newly weaned rats are susceptible to adverse effects including mortality on an immunosuppressed 200 ppm diet. The number of worms recovered from the body cavity increased as the study necropsy day was extended. Lung digestion assisted in recovery of further worms.

A third study was conducted to evaluate drug efficacy of selected compounds, with compounds and dosages selected based on efficacy from prior ferret and dog studies in the D. immitis rat model; and to determine the effect of withdrawal of immunosuppression at Day 70. Male CD (Sprague Dawley) IGS rats weighing 150-175 grams were randomized into specific treatment groups (n=6/group). On Day −8, medicated hydrocortisone diet at 200 ppm was provided ad-libitum to all treatment groups. On Day 0, infected larvae were harvested from Aedes aegypti approximately 15-17 days following membrane feeding on heparinized blood containing 50-100 microfilariae/20 μl. Rats were inoculated with approximately 50 (2×25) L3 D. immitis larvae (Michigan strain) by subcutaneous injection into the inguinal area. On Day 21, all animals had the 200 ppm hydrocortisone diet removed and replaced with 50 ppm hydrocortisone diet. On Day 30, all groups received a treatment medication (moxidectin (MOX), ivermectin (IVM), emodepside (EMO), a bisimide, a cyclooctoadepsipeptide, or an isoxazoline) by subcutaneous injection. On Days 34 and 38, the T09, T10 and T11 groups received further subcutaneous injections of the treatment medication. On Day 70, group T13 received un-medicated diet until necropsy. On Day 112 (±1) post-inoculation, all animals were necropsied for the presence of adult D. immitis worms. A blood sample was taken at necropsy for D. immitis antigen ((Ag); Ag+ (positive)). A lung digestion procedure was conducted to collect additional worms. Treatment medications were dosed by subcutaneous injection. The study design is shown below in Table 3 and the study results are described in Tables 4 and 5.

TABLE 3
Study design for the efficacy of various compounds
in the D. immitis immunosuppressed rat model

| | | | | | | Necropsy |
|---|---|---|---|---|---|---|
| | | | | | | day post- |
| | | | | Days of | Days of | inocu- |
| | Treat- | Dose | Dosing | 200 ppm | 50 ppm | lation |
| Group | ment | (mg/kg) | day(s) | diet | diet | (±1) |
| T01 | Control | 0 | 30 | −8 to 21 | 21-112 | 112 |
| T02 | IVN | 0.001 | 30 | −8 to 21 | 21-112 | 112 |
| T03 | IVN | 0.003 | 30 | −8 to 21 | 21-112 | 112 |
| T04 | MOX | 0.001 | 30 | −8 to 21 | 21-112 | 112 |
| T05 | MOX | 0.003 | 30 | −8 to 21 | 21-112 | 112 |
| T06 | EMO | 5 | 30 | −8 to 21 | 21-112 | 112 |
| T07 | EMO | 1 | 30 | −8 to 21 | 21-112 | 112 |
| T08 | Bisamide | 10 | 30 | −8 to 21 | 21-112 | 112 |
| T09 | Depsi-1 | 10 | 30, 34 | −8 to 21 | 21-112 | 112 |
| | | | and 38 | | | |
| T10 | Depsi-2 | 10 | 30, 34 | −8 to 21 | 21-112 | 112 |
| | | | and 38 | | | |
| T11 | Depsi-3 | 10 | 30, 34 | −8 to 21 | 21-112 | 112 |
| | | | and 38 | | | |
| T12 | Iso- | 30 | 30 | −8 to 21 | 21-112 | 112 |
| | xazoline | | | | | |
| T13 | Control W | 0 | N/A | −8 to 21 | 21-70 | 112 |
| T14 | Control L | 0 | N/A | −8 to 21 | 21-112 | 112 |
| T15 | Control | 0 | N/A | −8 to 21 | 21-112 | 120 |
| T16 | Control | 0 | N/A | −8 to 21 | 21-112 | 128 |

Depsi (1-3) = Novel cyclooctadepsipeptides
Control L (T14) = Lung digestion only

On Day −8, medicated hydrocortisone diet at 200 ppm was provided to all treatment groups. On Day 0, infected larvae were harvested from Aedes aegypti approximately 15-17 days following membrane feeding on heparinized blood containing 50-100 microfilariae/20 μl. Rats were inoculated with approximately 50 (2×25) L3 D. immitis larvae (Michigan strain) by subcutaneous injection into the inguinal area. On Day 21, all animals had the 200 ppm hydrocortisone diet removed and replaced with 50 ppm hydrocortisone diet. On Day 30, all groups received a treatment medication (moxidectin, ivermectin, emodepside, a bisimide, a cyclooctoadepsipeptide, or an isoxazoline) by subcutaneous injection. On Days 34 and 38, the T09, T10 and T11 groups received further subcutaneous injections of the treatment medication. On Day 70, group T13 received un-medicated diet until necropsy. On Day 112 (±1) post inoculation, all animals were necropsied for the presence of D. immitis. A blood sample was also taken at necropsy for D. immitis antigen. A lung digestion procedure was also conducted to collect additional worms.

TABLE 4
Arithmetic mean heartworm counts and % efficacy in rats

| | | | Dosing | Necropsy | Mean | # rats | | |
|---|---|---|---|---|---|---|---|---|
| | | | day(s) | day | worm | with | | |
| Group | | Dose | post- | post- | count | infection | # of | % |
| (n = 6) | Treatment | (mg/kg) | inoculation | inoculation | (range) | (n = 6) | Ag+ | Efficacy |
| T01 | Control | 0 | 30 | 112-114 | 7.5 | 6 | 2 | N/A |
| | | | | | (5-13) | | | |
| T02 | IVM | 0.001 | 30 | 112-114 | 0.8 | 5 | 2 | 89.3 |
| | | | | | (0-2) | | | |
| T03 | IVM | 0.003 | 30 | 112-114 | 0.3 | 1 | 2 | 96.5 |
| | | | | | (0-2) | | | |
| T04 | MOX | 0.001 | 30 | 112-114 | 0.67 | 4 | 1 | 91.7 |
| | | | | | (0-1) | | | |
| T05 | MOX | 0.003 | 30 | 112-114 | 0 | 0 | 0 | 100 |
| T06 | EMO | 5.00 | 30 | 112-114 | 0 | 0 | 1 | 100 |
| T07 | EMO | 1.00 | 30 | 112-114 | 1.8 | 3 | 1 | 75.6 |
| | | | | | (2-5) | | | |
| T08 | Bisamide | 10 | 30 | 112-114 | 5.8 | 6 | 3 | 24.6 |
| | | | | | (2-10) | | | |
| T09 | Depsi-1 | 10 | 30, 34 | 112-114 | 0 | 0 | 0 | 100 |
| | | | and 38 | | | | | |
| T10 | Depsi-2 | 10 | 30, 34 | 112-114 | 0 | 0 | 2 | 100 |
| | | | and 38 | | | | | |
| T11 | Depsi-3 | 10 | 30, 34 | 112-114 | 0 | 0 | 2 | 100 |
| | | | and 38 | | | | | |
| T12 | Isoxazoline | 30 | 30 | 112-114 | 5.3 | 6 | 4 | 28.9 |
| | | | | | (2-11) | | | |

Table 4 demonstrates that rats from the control (T01) group had an infection with an average of 7.5 worms per rat. The ivermectin (IVM) and moxidectin (MOX) groups achieved a similar dose efficacy response of 89.3% and 96.5% for IVM at 0.001 and 0.003 mg/kg and 91.7% and 100% for moxidectin at the same doses, respectively. Emodepside also achieved a dose response percent efficacy of 75.6 and 100 at 1.0 and 5.0 mg/kg, respectively. The three cyclooctadepsipeptides dosed at 10 mg/kg on days 30, 34 and 38 all achieved 100% efficacy. The bisimide and isoxazoline administered at 10 and 30 mg/kg were ineffective, achieving 24.6% and 28.9% efficacy, respectively.

Overall, the study achieved comparable efficacies to compounds that have been dosed in dogs and/or ferrets. Antigen (DiroCHECK) tests were not fully predictive of worm infection; possibly because the test only detects tiny pieces of female (not male) heartworm skin proteins circulating in the blood.

TABLE 5
Arithmetic mean heartworm counts in rats

| | | Necropsy | Mean | # | # |
|---|---|---|---|---|---|
| | | day | worm | rats with | recovered |
| Group | | post- | count | infection | by |
| (n = 6) | Treatment | inoculation | (range) | (n = 6) | digestion |
| T13 | Control (medicated | 112-114 | 4.5 | 6 | Not |
| | diet withdrawal | | (1-8) | | Recorded |
| | day 70) | | | | |
| T14 | Control (lung | 112-114 | 4.5 | 6 | *12 |
| | digest) | | (2-7) | | |
| T15 | Control (extend | 120 | 8.2 | 6 | 3 |
| | +8 days) | | (3-13) | | |
| T16 | Control (extend | 128 | 3.8 | 6 | |
| | +16 days) | | (2-10) | | |

*No dissection of lung employed

Table 5 shows that substitution of the medicated diet at Day 70 with un-medicated (T13) moderately decreased worm burden as did worm recovery by lung digestion (T14) when compared to T13. Extending the duration of infection to 120 (T15) and 128 (T16) days after inoculation achieved slightly increased and decreased worm burden means, respectively. All control rats had worms and no adverse effects were observed throughout the study. Lung digestion was shown to recover low numbers of additional worms and was best employed as a contributory procedure to dissection.

A fourth study was conducted to determine the effect of immunosuppressed diet withdrawal at strategic life stages of D. immitis infection in rats. Male CD (Sprague Dawley) IGS rats weighing 150-175 grams were used for this study (n=6/group). On Day −8, medicated hydrocortisone diet at 200 ppm was provided to all treatment groups, ad libitum. On Day 0, L3 were harvested from A. aegypti approximately 15-17 days following membrane feeding on heparinized blood containing 50-100 microfilariae/20 μl. Rats were inoculated with approximately 60 (2×30) L3 D. immitis larvae by subcutaneous injection into the inguinal area. On Day 21, all 200 ppm hydrocortisone diets were replaced with a 50 ppm hydrocortisone diet. On Day 31, cages 1, 2 and 3 had the 50 ppm hydrocortisone diet removed and replaced with standard rat non-medicated diet. On Day 45, cages 4, 5 and 6 had the 50 ppm hydrocortisone diet removed and replaced with standard rat non-medicated diet. On Day 62, cages 7, 8, 9 and 10 had the 50 ppm hydrocortisone diet removed and replaced with standard rat non-medicated diet. All rats were necropsied on Day 119 (±1) post-inoculation. The study design is presented in Table 6 and the study data is presented in Table 7.

TABLE 6
Study design for diet withdrawal

| | | | | | Necropsy | | |
|---|---|---|---|---|---|---|---|
| | | | Days of | Days of | day (±1) | | |
| | | Day of | 200 ppm | 50 ppm | post- | | # of |
| Group | Treatment | inoculation | diet | diet | inoculation | Cage # | rats |
| T01 | Control | 0 | −8 to 21 | 21-31 | 119 | 1, 2, 3 | 6 |
| T02 | Control | 0 | −8 to 21 | 21-45 | 119 | 4, 5, 6 | 6 |
| T03 | Control | 0 | −8 to 21 | 21-62 | 119 | 7, 8, 9, 10 | 8 |

TABLE 7
Mean worm counts with varied diet withdrawal

| | Day of diet | Necropsy | | | | |
|---|---|---|---|---|---|---|
| | withdrawal | day | | Live | Arith- | Geo- |
| | (post- | (post- | | worm | metic | metric |
| Group | inoculation) | inoculation) | Animal | count | mean | mean |
| T01 | 31 | 119 | 1 | 0 | 0 | 0 |
| | | | 2 | 0 | | |
| | | | 3 | 0 | | |
| | | | 4 | 0 | | |
| | | | 5 | 0 | | |
| | | | 6 | 0 | | |
| T02 | 45 | 119 | 7 | 1 | 3.17 | 1.91 |
| | | | 8 | 2 | | |
| | | | 9 | 5 | | |
| | | | 10 | 0 | | |
| | | | 11 | 0 | | |
| | | | 12 | 11 | | |
| T03 | 62 | 119 | 13 | 2 | 4.25 | 2.85 |
| | | | 14 | 1 | | |
| | | | 15 | 3 | | |
| | | | 16 | 6 | | |
| | | | 17 | 0 | | |
| | | | 18 | 15 | | |
| | | | 19 | 5 | | |
| | | | 20 | 2 | | |

On 3 separate previous occasions (all data not shown), using 12 rats in total, withdrawal of immunosuppressed diet on Day 70 post-inoculation demonstrated little or no effect on worm burdens. Overall, reducing immunosuppression to the minimum level required is beneficial to the general health status of the animals on study and potentially reduces any effect on treatments administered following medicated diet withdrawal.

Data from numerous studies were compiled to correlate dose data from rat and dog. As can be seen in Table 8, the efficacy achieved between rat and dog at recommended (approved) macrocyclic lactone (ML) doses for dog were generally comparable in rat. Dogs were inoculated with approximately 50 D. immitis L3 larvae (UGA (University of Georgia) susceptible strain) and were orally administered the ML or emodepside at 1, 1.5, and/or 2 months post-inoculation. Dogs were necropsied at about 5 months post-inoculation. Rats were inoculated with approximately 50 D. immitis L3 larvae (Michigan susceptible strain) and were administered the ML or emodepside by subcutaneous injection at 1 month post-inoculation. Rats were necropsied at about 4 months post-inoculation. As can be observed in Table 8, the efficacy between the subcutaneous dosing in the rat and oral dosing in the dog models correlated well. Thus, the efficacy of antifilarial drugs in the rat model correlates well with efficacy in the dog model. Studies are ongoing to directly compare oral dosing in the rat versus subcutaneous dosing using susceptible heartworm strains. It was also found that the D. immitis life cycle in the immuno-suppressed rat was about 1 month shorter than in dogs. This time savings provides an additional reduction in housing cost of the animal colony.

TABLE 8
Correlation of drug efficacy in rat (SQ) and dog (oral)
heartworm (D. immitis) models

| Approved oral | Oral dose | SQ dose | Month(s) post- | |
|---|---|---|---|---|
| dose in | tested in dog | tested in rat | inoculation | % |
| dog (μg/kg) | (μg/kg) | (μg/kg) | dosed | Efficacy |
| Moxidectin | 1.25 | — | 1.2 | 100 |
| (3) | 0.625 | — | 2 | 100 |
| | 0.5 | — | 2 | 100 |
| | — | 1 | 1 | 92 |
| | — | 3 | 1 | 100 |
| Ivermectin | 1 | — | 1 | 53 |
| (6) | 2 | — | 1 | 83-97 |
| | 3.3 | — | 1 | 98 |
| | 6 | — | 1 | 100 |
| | 2 | — | 1.5 | 64 |
| | 6 | — | 1.5 | 100 |
| | — | 1 | 1 | 89 |
| | — | 3 | 1 | 97-100 |
| | — | 6 | 1 | 100 |
| Emodepside | 1 | — | 1 | 58 |
| (N/A) | 3 | — | 1 | 88 |
| | 5 | — | 1 | 97 |
| | 10 | — | 1 | 97 |
| | — | 1 | 1 | 76 |
| | — | 5 | 1 | 100 |

In yet another study, moxidectin was used to treat immunosuppressed CD (Sprague Dawley) IGS male rats (250-350 g) infected with 2 characterized macrocyclic lactone resistant D. immitis strains to assess efficacy and plasma exposure. Rats were inoculated with L3 larvae of ZoeMI-01 (ZM1) and JYD-34 D. immitis strains. T01 and T06 were control (C) groups. Animals received either a 3, 12, or 24 μg/kg dose of moxidectin 28 days post L3 inoculation. In addition, two groups, T05 and T10, received 3 μg/kg doses 28, 56, and 84 days post L3 inoculation. Moxidectin was administered by oral gavage. Animals were necropsied 120 days post L3 inoculation and worms were harvested. Data for this efficacy study is provided in Table 9.

TABLE 9
Geometric Mean Worm Counts and Efficacy in Rats

| | | | | Post- | | | | |
|---|---|---|---|---|---|---|---|---|
| | | | Oral | inoculation | Necropsy | Mean | Number | |
| Group | | | dose | dosing | day (post- | worm | of rats | % |
| (n = 4) | Strain | Treatment | (μg/kg) | (day(s)) | inoculation) | counts | infected | efficacy |
| T01 | ZM1 | C | 0 | 28 | 120 | 5.6 | 4 | N/A |
| T02 | ZM1 | MOX | 3 | 28 | 120 | 1.1 | 3 | 81 |
| T03 | ZM1 | MOX | 12 | 28 | 120 | 0 | 0 | 100 |
| T04 | ZM1 | MOX | 24 | 28 | 120 | 0 | 0 | 100 |
| T05 | ZM1 | MOX | 3 | 28, 56, 84 | 120 | 0 | 0 | 100 |
| T06 | JYD-34 | C | 0 | 28 | 120 | 3.9 | 4 | N/A |
| T07 | JYD-34 | MOX | 3 | 28 | 120 | 6.0 | 4 | 0 |
| T08 | JYD-34 | MOX | 12 | 28 | 120 | 2.8 | *3 | 28 |
| T09 | JYD-34 | MOX | 24 | 28 | 120 | 0.5 | 1 | 87 |
| T10 | JYD-34 | MOX | 3 | 28, 56, 84 | 120 | 2.4 | 4 | 38 |

*One rat died pre-necropsy

Moxidectin administered by oral gavage at 28 days post infection against a macrocyclic lactone (ML) susceptible D. immitis strain (ZM1; T-02) at 3 μg/kg achieved 81% efficacy. When administered similarly at 12 and 24 μg/kg at 28 days post infection and at 28, 56, and 84 days post infection at 3 μg/kg 100% efficacy was achieved. Moxidectin administered by oral gavage at 28 days post infection against a JYD ML-resistant D. immitis strain at 24 μg/kg was 87% efficacious. Overall, the efficacy and plasma exposure of oral moxidectin using varying dosing regimens, against two strains of D. immitis, demonstrated that the rat model can accurately evaluate in vivo drug susceptibility of ML-resistant strains that correlate with similar results in the dog. Additional studies are being planned to evaluate more compounds and strains.

D. immitis Adult (Immature and Mature) Worm Assays (In Vitro and In Vivo)

Adult heartworms were obtained from rat heart and lungs aseptically following euthanasia. The adult worms were maintained in a general purpose cell culture media for about 7-10 days at 37° C. For the in vitro assay, test compounds were dissolved and serially diluted in dimethylsulfoxide (DMSO). Aliquots were added to the empty wells of assay plates. The cell culture media and a single adult (immature/mature) D. immitis worm was added to each well to dilute the test compounds to the desired concentrations. Assay plates were incubated for approximately 24 hours, and the worms in the assay wells were observed visually for drug effect. Worms were assessed subjectively for survival or paralysis, and results were reported as Minimum Effective Dose (MED). Data for these assays are not reported.

For future in vivo studies, immature adult heartworms extracted from immunosuppressed rats will be surgically or directly transferred into the dogs' circulatory system. These studies, if successful, will reduce or eliminate the need for dog donors of adult heartworms for experimental studies. These recipient dogs will be monitored for microfilaria levels and adult heartworm antigen to determine the success of the transplant procedures from rat to dog.

We claim:

1. An immunosuppressed laboratory animal model for dirofilarial heartworm nematodes wherein said animal is fed a dietary admixture of an immunosuppressing agent before and after inoculation of dirofilarial L3 larvae.

2. The animal model according to claim 1, wherein the immunosuppressing agent is a glucocorticoid.

3. The animal model according to claim 2, wherein the laboratory animal is a rodent.

4. The animal model according to claim 1, wherein the dirofilarial heartworm nematode is Dirofilaria immitis or Dirofilaria repens.

5. The animal model according to claim 2, wherein the glucocorticoid is hydrocortisone or a salt thereof.

6. The animal model according to claim 5, wherein the glucocorticoid is hydrocortisone acetate and the rodent is a rat.

7. The animal model according to claim 6, wherein the dietary admixture comprises about 20 ppm to about 250 ppm hydrocortisone.

8. The animal model according to claim 7, wherein the dietary admixture comprises about 50 ppm or about 200 ppm hydrocortisone.

9. The animal model according to claim 8, wherein the rat is fed the dietary admixture comprising about 200 ppm hydrocortisone for at least three days before inoculation with Dirofilaria immitis L3 larvae.

10. The animal model according to claim 9, wherein the rat is fed the dietary admixture comprising about 200 ppm hydrocortisone for about 8 days before inoculation and then for about 8 to about 14 days after inoculation.

11. The animal model according to claim 10, wherein the rat is fed the dietary admixture comprising about 200 ppm hydrocortisone on the day of L3 inoculation and for about 12 days after L3 inoculation.

12. The animal model according to claim 11, wherein the rat is fed the dietary admixture comprising about 200 ppm hydrocortisone for about 8 days prior to L3 inoculation, fed the same amount of hydrocortisone on the day of L3 inoculation and fed the same amount of hydrocortisone for about 12 days after L3 inoculation totaling about 21 days and then fed a dietary admixture comprising about 50 ppm hydrocortisone through necropsy.

13. The animal model according to claim 12, wherein the rat is fed the dietary admixture comprising about 50 ppm hydrocortisone for at least 70 days through necropsy.

14. A method for preparing an immunosuppressed rodent for a dirofilarial heartworm nematode animal model comprising:
a. administering about 200 ppm of a glucocorticoid in a dietary feed admixture for at least 3 days;
b. inoculating the rodent with dirofilarial L3 larvae;
c. administering about 200 ppm of a glucocorticoid in a dietary feed admixture for at least 3 days post-inoculation; and subsequently
d. administering about 50 ppm of a glucocorticoid in a dietary feed admixture for at least 70 days through necropsy.

15. The method according to claim 14, wherein the rodent is a rat and the glucocorticoid is hydrocortisone, or salt thereof.

16. The method of claim 15, wherein the hydrocortisone is hydrocortisone acetate and the dirofilarial larvae is a Dirofilaria immitis or Dirofilaria repens larvae.

17. The method of claim 14 for preparing an immunosuppressed rodent for a dirofilarial heartworm nematode animal model comprising:
a. administering about 200 ppm of a glucocorticoid in a dietary feed admixture for about 5 to about 10 days;
b. inoculating the rodent with dirofilarial L3 larvae wherein the L3 larvae are Dirofilaria immitis or Dirofilaria repens larvae;
c. administering about 200 ppm of a glucocorticoid in a dietary feed admixture for about 5 to about 20 days post-inoculation; and subsequently
d. administering about 50 ppm of a glucocorticoid in a dietary feed admixture for at least 70 days through necropsy; and
e. wherein the glucocorticoid is hydrocortisone, or salt thereof, and the rodent is a rat.

18. The method of claim 17 wherein the rat is administered about 200 ppm hydrocortisone for about 7 to about 9 days, inoculated with dirofilarial L3 larvae, administered about 200 ppm hydrocortisone for about 8 to about 14 days after L3 inoculation and then administered about 50 ppm hydrocortisone for at least 70 days through necropsy.

19. The method of claim 18 wherein dirofilarial L3 larvae is a Dirofilaria immitis larvae.

20. The method of claim 18 wherein the dirofilarial larvae is a Dirofilaria repens larvae.
